(19) United States
(12) Patent Application Publication
(10) Pub. No.: US 2002/0143295 A1
(43) Pub. Date: Oct. 3, 2002

(54) Article of manufacture for intraveneous access and process for making it saver with the, ''safe cath''

(76) Inventors: Meritta D. Taylor, Orange, TX (US); Russell Milligan, Orange, TX (US)

Correspondence Address: Meritta D. Taylor, 811 Dayton, Orange, TX, 77630, US

(21) Appl. No.: 09/824,387

(22) Filed: Apr. 2, 2001

(51) Int. Cl. A61M 5/178

(52) U.S. Cl. 604/165.03

(57) ABSTRACT

An article of manufacture for intraveneous access with; An angiocath with an adhesive attached to the anchor for proper placement, and A intravenous catheter inserted into the human vein with peel off adhesive to prevent injury.

### BACKGROUND OF THE INVENTION

This invention relates generally to the field of Science and Medicine, and more particularly to an article of manufacture for intraveneous access and process for making it saver, cost efficient, and less tramatic for the patients.

Since the conception of intravenous insertion, there has not been a product on the market to aide in the insertion of a needle into the human vein.

Today there has still be no changes made to the present way of intravenous insertion. The way a catheter is inserted into the human vein presently is deficient because far to many times it is unsuccessful due to the fact that there is no secured system in place to prevent this from happening.

### SUMMARY OF THE INVENTION

The primary object of the invention is To provide security after accessing an intravenous vein. in the human body.

Another object of the invention is To provide a more efficent way to secure a catheter into the vein.

Another object of the invention is To provide a cost effective way to secure a catheter into the vein.

A further object of the invention is To provide a less tramatic way of securing a catheter into the vein.

Other objects and advantages of the present invention will become apparent from the following descriptions, taken in connection with the accompanying drawings, wherein, by way of illustration and example, an embodiment of the present invention is disclosed.

An article of manufacture for intraveneous access comprising: An angiocath with an adhesive attached to the anchor for proper and saver placement, and an intravenous catheter inserted into the human vein with peel off adhesive to prevent injury.

A process for intraveneous access comprising the steps of: An angiocath with an adhesive attached to the anchor for proper placement, and an intravenous catheter inserted into the human vein with peel off adhesive to prevent injury.

With this modification of the present intraveneous catheter, doctors and nurses will be able to insert an intraveneous catheter into the vein of an adult or child with confidence.

With the present way of insertion, strips of tape are pulled a placed on the side of the bed, IV pole, or where ever one can put it to have access when need. This procedure is risky because once the catheter is in the vein you have to reach for the tape to anchor the hub down and take a chance of losing the vein, esp., with children., not to mention the adhesive you now have to get off of the bedside or pole, which is a time waster, which enturn takes time away from the patient.

The drawings constitute a part of this specification and include exemplary embodiments to the invention, which may be embodied in various forms. It is to be understood that in some instances various aspects of the invention may be shown exaggerated or enlarged to facilitate an understanding of the invention.

### BRIEF DESCRIPITION OF THE DRAWINGS

FIG. 1 is a perspective view of the modified angiocath next to the regular angiocath.

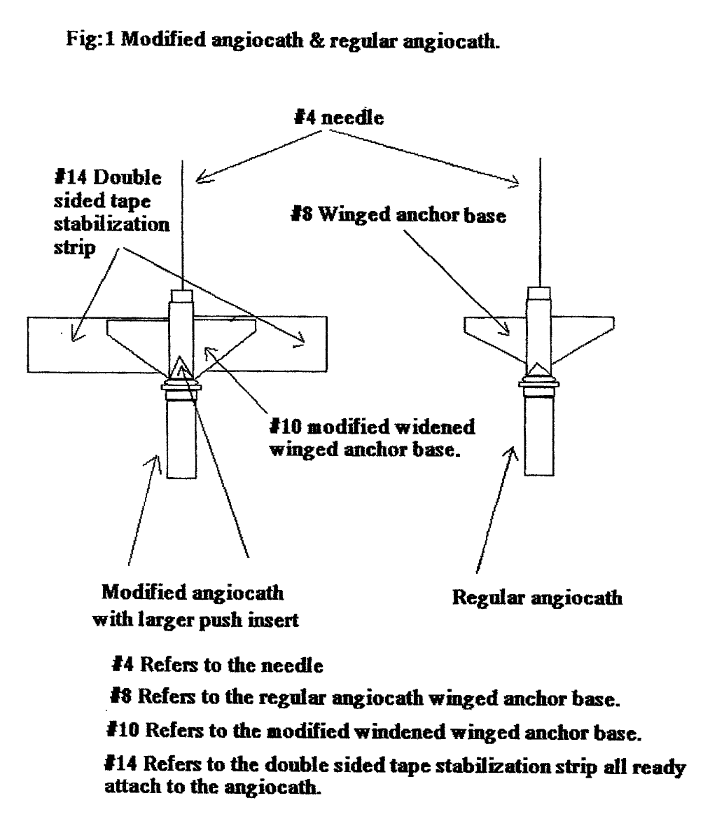

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

An article of manufacture for intraveneous access and process to make it more effcient, saver way to access the vein.

This product consist of an intraveneous canula that has an adhesive attached for secured placement. As previously mentioned, the present procedure for the insertion of an intraveneous catheter in the human vein for an adult or child is deficient. The current procedure has the insertion catheter separate from the other supplies needed such as the tape or adhensive. With my modification, the product will contain adhesive directly on the insertion catheter preventing the person from fumbling around trying to retreve the tape in order to secure it, and then trying to place it under the hub in order to anchor it. Can you imagine preforming this task on a small child that is screaming and fighting you the whole time!

As a nurse, and many nurses I have spoken with concerning this problem, this product would be an asset and well excepted.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. An article of manufacture for intraveneous access comprising:
An angiocath with an adhesive attached to the anchor for proper placement; and
A intravenous catheter inserted into the human vein with peel off adhesive to prevent injury.

2. A process for intraveneous access comprising the steps of:
An angiocath with an adhesive attached to the anchor for proper placement; and
A intravenous catheter inserted into the human vein with peel off adhesive to prevent injury.
